(19) United States
(12) Patent Application Publication
(10) Pub. No.: US 2007/0249995 A1
(43) Pub. Date: Oct. 25, 2007

(54) Medical Instrument For Suction And Irrigation

(76) Inventor: Agnes Maria Van Manen, 's - Hertogenbosch (NL)

Correspondence Address: ST. ONGE STEWARD JOHNSTON & REENS, LLC, 986 BEDFORD STREET, STAMFORD, CT, 06905-5619, US

(21) Appl. No.: 11/558,734

(22) Filed: Nov. 10, 2006

(30) Foreign Application Priority Data
Nov. 11, 2005 (DE) 10 2005 055 326.5

(51) Int. Cl. A61M 31/00

(52) U.S. Cl. 604/93.01

(57) ABSTRACT

The invention relates to a medical instrument for suction and irrigation, comprising at least two connectors having a body comprising a channel for the passage of a fluid. The at least connectors comprise a tactile arrow-like portion that indicates a direction of flow of the fluid through the channel, whereby a tactile arrow-like portion of one of the at least two connectors indicates a direction of flow towards the medical instrument, and a tactile arrow-like portion of another one of the at least two connectors indicates a direction of flow away from the medical instrument.

### BACKGROUND OF THE INVENTION

The invention relates to a medical instrument for suction and irrigation which comprises at least two connectors having a body comprising a channel for the passage of a fluid.

Medical instruments for suction and irrigation are known and are sold for example by the applicant for use in laparoscopy.

These instruments serve for example for the purpose of cleaning a viewing window of an endoscope of blood or other body fluids during an endoscopic examination or treatment, or of removing blood and body fluids from an operating site.

It is thereby important that the connecting lines are always attached to the correct connectors, since otherwise for example body fluids get into the irrigation portion of an instrument for suction and irrigation that has to be kept sterile, and can possibly also contaminate the sterile irrigation fluid.

Previously, visual information has been provided on such instruments in the form of arrows or lettering. However, this has the disadvantage that the surgeon may have to look up from the operating site and cannot detect with other senses whether a connector and a valve possibly attached to it is an irrigation connector or a suction connector.

Furthermore, certain operations, in particular those in which fluorescence phenomena are used, are carried out in a dark operating theatre, which makes the detection of visual information much more difficult again.

It is therefore one object of the invention to define a medical instrument for suction and irrigation having at least two connectors by means of which the direction of flow of a fluid through these connectors can be detected quickly and without visual information.

### SUMMARY OF THE INVENTION

According to the invention, this object is achieved by a medical instrument comprising at least two connectors having a tactile arrow-like portion that indicates a direction of flow of the fluid through the channel, whereby a tactile arrow-like portion of one of the at least two connectors indicates a direction of flow towards the medical instrument and a tactile arrow-like portion of another one of the at least two connectors indicates a direction of flow away from the medical instrument.

A tactile arrow-like portion is understood here as meaning any construction by means of which by touch alone directional information can be conveyed. Examples of this are the arrangement of ribbed engravings provided transversely in relation to the direction of flow, which converge in one direction, similarly formed elevations, shapes in the form of an arrow, triangle, pyramid, cone or the like arranged on the connector in such a manner that they can be felt, or else a series of objects of decreasing size, such as for example a series of beads becoming smaller in the direction of flow.

In an embodiment, the tactile arrow-like portion is arranged on the body, in particular the tactile arrow-like portion is formed by the body.

As a result of this measure, the directional indicator is located directly at the connector and thereby makes a quick detection of the direction of flow possible. If the body forms the tactile arrow-like portion, this also makes production easier, since the tactile arrow-like portion can be formed directly during the production of the body.

In a further embodiment of the invention, at least one of the at least two connectors comprises a valve.

As a result of this measure, the function of opening and closing a flow through the connector is directly associated with the connector. There is consequently a link between the control of the flow and the information on the direction of the flow.

In an embodiment of the aforementioned measure, the tactile arrow-like portion is arranged on the valve and, in particular, is formed by the valve.

As a result of this measure, the directional information is directly accessible at the location at which the supply or removal of a fluid is initiated. During the process of taking hold of the valve in order to open or close it, the direction of flow of this valve is thereby detected almost automatically, which again contributes significantly to the safety of operating the instrument.

In a further embodiment of the invention, the tactile arrow-like portion comprises at least a first region and a second region, the second region being arranged downstream of the first region in the direction of flow and having a smaller width than the first region in at least one plane along the direction of flow.

It is hereby understood that the expressions “first region” and “second region” only serve to distinguish the two regions from each other and shall not be interpreted as indicating any particular order or preference.

As a result of such an arrangement, a structure which tapers in the direction of flow and can be detected by touch is formed. In this embodiment the tactile arrow-like portion can be detected by touch particularly easily.

In an embodiment of the aforementioned measure, the tactile arrow-like portion tapers continuously from the first region to the second region.

In the case of a discontinuous taper, accurate information on the direction of flow cannot be obtained at certain locations. In the case of a continuous taper, by contrast, the directional information can be acquired by touch at any point of the tactile arrow-like portion in a simple way.

In an embodiment of the aforementioned measure, the tactile arrow-like portion forms an arrow.

The design of the tactile arrow-like portion as an arrow forms a particularly intuitive way of conveying information.

In a further embodiment of the invention, the tactile arrow-like portion is formed by an engraving.

An engraving has the advantage that it can be provided on a connector in a structurally simple manner. It also has the advantage that even already existing instruments can in this way be provided with a tactile arrow-like portion according to the invention in a simple manner.

In a further embodiment of the invention, the tactile arrow-like portion is formed by an elevation.

An elevation has the advantage that it can be formed during the forming of the connector in a simple manner in production engineering terms. This measure also has the advantage that elevations can be sensed by touch particularly easily.

It goes without saying that the features mentioned above and still to be explained below can be used not only in the combinations specified but also in other combinations or on their own without departing from the scope of the present invention.

### BRIEF DESCRIPTION OF THE DRAWINGS

The invention is now further explained in more detail by way of example on the basis of the accompanying drawings, in which:

FIG. 1 shows a perspective view of a first connector for a medical instrument for suction and irrigation;

FIG. 2 shows a perspective view of a second connector for a medical instrument for suction and irrigation;

FIG. 3 shows a side view of a third connector for a medical instrument for suction and irrigation;

FIG. 4 shows a side view of a fourth connector for a medical instrument for suction and irrigation; and

FIG. 5 shows a medical instrument for suction and irrigation.

### DETAILED DESCRIPTION OF PREFERRED EMBODIMENTS

In FIG. 1, a connector is denoted in its entirety by the reference numeral 10.

The connector 10 comprises a body 12, through which a channel 14 runs. Via this channel 14, a fluid can be passed through the connector 10.

The body 12 comprises a first end 16, at which a connecting piece 18 is arranged. With this connecting piece 18, the connector 10 can be connected to a line for a fluid.

The body 12 further comprises a second end 20, at which the connector 10 can be connected to a medical instrument for suction and irrigation.

Arranged between the first end 16 and the second end 20 is a tactile arrow-like portion 22. This tactile arrow-like portion 22 comprises a first region 24 and a second region 26. The first region 24 has a greater width than the second region 26 in a plane along the direction of flow, i.e. along the channel 14. The tactile arrow-like portion 22 tapers continuously from the first region 24 to the second region 26. As a result, the tactile arrow-like portion 22 has approximately the form of a triangle and, together with the body 12, forms the shape of an arrow.

Since the first region 24 is located here to the left of the second region 26, it is indicated that the fluid is being passed through the connector 10 from the first end 16 in the direction of an arrow 28 to the second end 20.

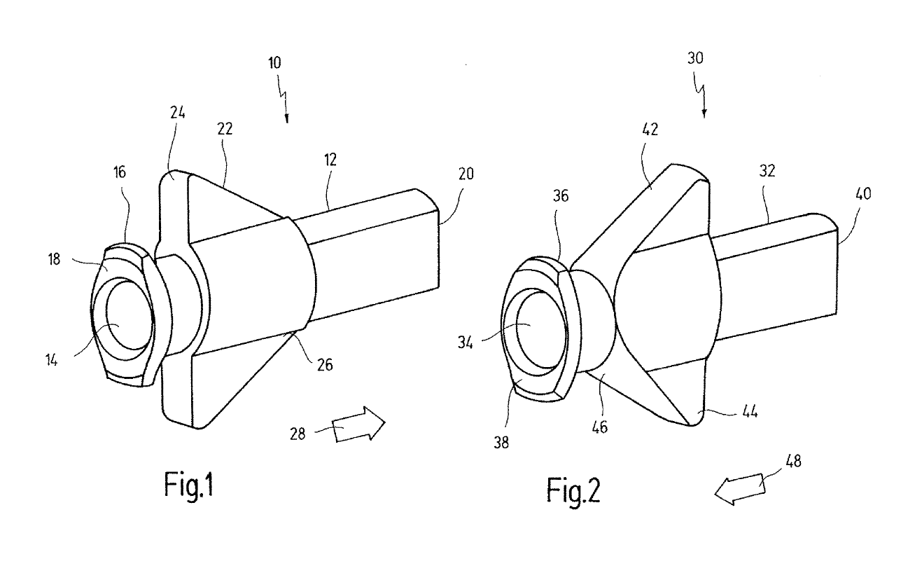

In FIG. 2, a second connector is denoted by the reference numeral 30.

The connector 30 is of substantially the same construction as the connector IO and forms the component complementing the latter.

The connector 30 comprises a body 32, through which a channel 34 runs. Via this channel 34, a fluid can be passed through the connector 30.

The body 32 comprises a first end 36, at which a connecting piece 38 is arranged. With this connecting piece 38, the connector 32 can be connected to a line for a fluid.

The body 32 further comprises a second end 40, at which the connector 30 can be connected to a medical instrument for suction and irrigation.

Arranged between the first end 36 and the second end 40 is a tactile arrow-like portion 42. This tactile arrow-like portion 42 comprises a first region 44 and a second region 46. The first region 44 has a greater width than the second region 46 in a plane along the direction of flow, i.e. along the channel 34. The tactile arrow-like portion 42 tapers continuously from the first region 44 to the second region 46. As a result, the tactile arrow-like portion 42 has approximately the form of a triangle and, together with the body 32, forms the shape of an arrow.

In this case, the first region 44 is arranged on the right-hand side and the second region 46 is arranged on the left-hand side. As a result, a direction of flow from the second end 40 towards the first end 36 in the direction of arrow 48 is indicated.

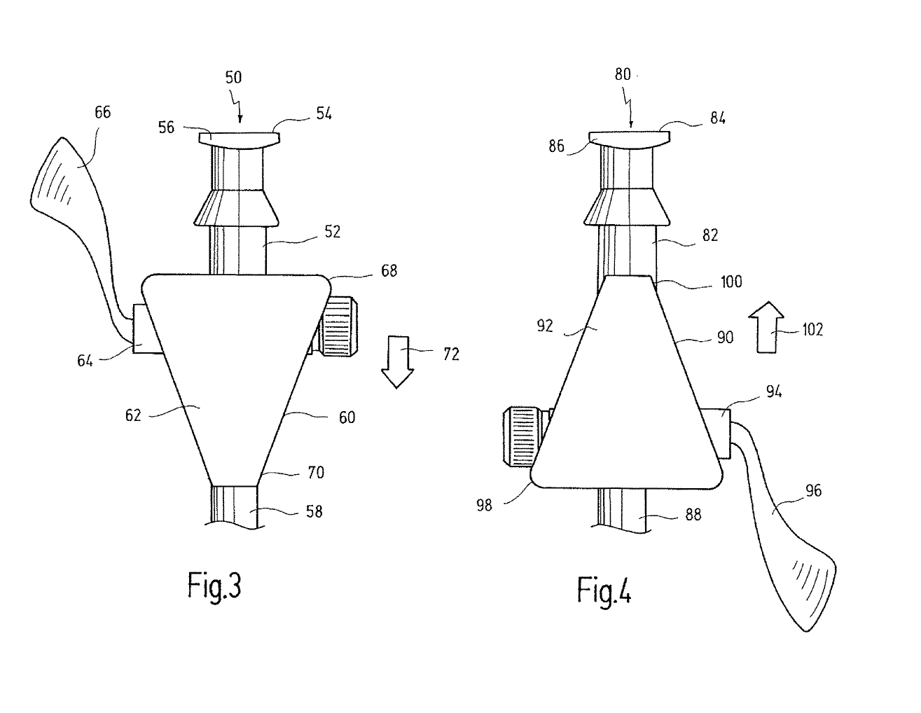

In FIG. 3, a third connector is denoted in its entirety by the reference numeral 50.

The connector 50 comprises a body 52, through which a channel not represented here runs.

The body 52 comprises a first end 54, at which a connecting piece 56 is arranged. By this connecting piece 56, the connector 50 can be connected to a line for a fluid, for example a supply line for an irrigation fluid.

The body 52 further comprises a second end 58, at which the connector 50 is connected to a medical instrument for suction and irrigation, not shown here.

Arranged between the first end 54 and the second end 58 is a valve 60. This valve 60 comprises a valve body 62 and a cock 64. The cock 64 has a lever 66, by means of which the valve 60 can be operated. If the lever 66 is arranged parallel to the body 52, the valve 60 is open, and a fluid can flow through the connector 50. If the lever 66 is angled away from this position, the valve 60 is closed.

The valve body 62 has a first region 68 and a second region 70. The first region 68 has a greater width than the second region 70 in a plane running along the direction of flow and tapers continuously from the first region 68 to the second region 70, giving the valve body 62 a substantially triangular form. This means here the valve body 62 forms the tactile arrow-like portion. It is indicated by this form that a fluid is flowing through the connector 50 from the first end 54 towards the second end 58, that is to say in the direction of arrow 72.

In FIG. 4, a fourth connector is denoted in its entirety by the reference numeral 80.

The connector 80 is of substantially the same construction as the connector 50 and forms the component complementing the latter.

The connector 80 comprises a body 82, through which a channel not represented here runs.

The body 82 comprises a first end 84, at which a connecting piece 86 is arranged. By this connecting piece 86, the connector 80 can be connected to a line for a fluid, for example a suction line.

The body 82 further comprises a second end 88, at which the connector 80 is connected to a medical instrument for suction and irrigation, not shown here.

Arranged between the first end 84 and the second end 88 is a valve 90. This valve 90 comprises a valve body 92 and a cock 94. The cock 94 has a lever 96, by means of which the valve 90 can be operated. If the lever 96 is arranged parallel to the body 82, the valve is open, and a fluid can flow through the connector 80. If the lever 96 is angled away from this position, the valve 90 is closed.

The valve body 92 comprises a first region 98 and a second region 100. The first region 98 has a greater width than the second region 100 in a plane running along the direction of flow and tapers continuously from the first region 98 to the second region 100, giving the valve body 92 a substantially triangular form. This means here the valve body 92 forms the tactile arrow-like portion. It is indicated by this form that a fluid is flowing through the connector 80 from the second end 88 towards the first end 84, that is to say in the direction of arrow 102.

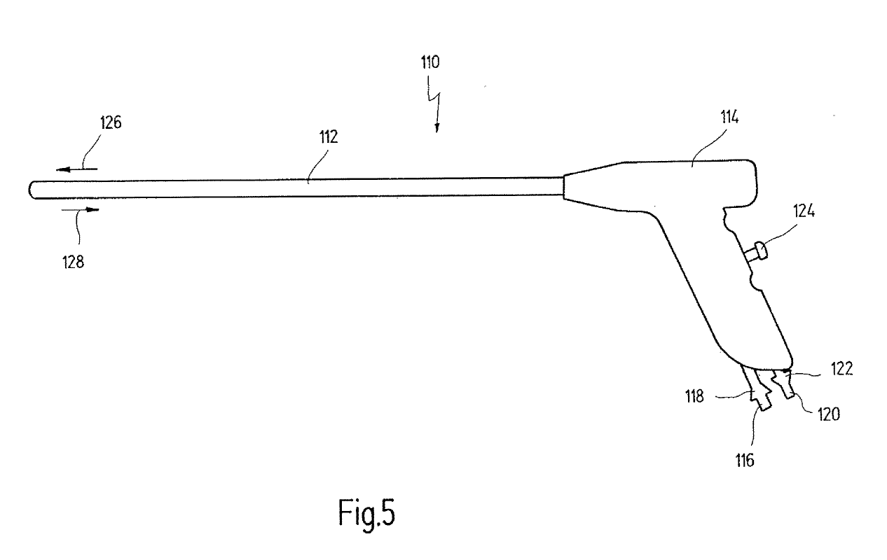

In FIG. 5, a medical instrument for suction and irrigation is denoted in its entirety by the reference numeral 110.

The instrument for suction and irrigation 110 comprises a shaft 11.2, with which an irrigation fluid can be introduced into the body of a patient and fluids can be removed from the body by suction.

Arranged at the proximal end of the shaft 112 is a handle 114, which here has the form of a pistol grip. Arranged on this handle 114 are a first connector 116 with a first tactile arrow-like portion 118 and a second connector 120 with a second tactile arrow-like portion 122.

The two connectors 116 and 120 serve to connect the instrument for suction and irrigation 110 to a supply line for an irrigation fluid and a suction line and to introduce fluids into the body of a patient and removing them from it by suction via the shaft 112.

The first tactile arrow-like portion 118 tapers towards the handle 114, thereby indicating that the supply line for the irrigation fluid is to be attached here. The second tactile arrow-like portion 122 tapers away from the handle 114, thereby indicating that the suction removal line is to be attached here. Through this design, it can be detected immediately and intuitively which lines are to be arranged at the connectors. This can possibly even take place without looking.

It is clear to a man of the art, that this tactile arrow-like portions 118 and 122 can be supplemented or replaced by other means detectable by touch such as engravings or elevations, for example, in the form of an arrow or one or more triangles indicating the direction of flow of a fluid through the connectors 116 and 120.

Also arranged on the handle 114 is the valve lever 124. The valve lever 124 can be moved upwards and downwards. The valve lever 124 blocks or releases the flow of the flushing fluid.

What is claimed is:

1. A medical instrument for suction and irrigation, comprising at least two connectors having a body comprising a channel for the passage of a fluid,
said at least two connectors having a tactile arrow-like portion that indicates a direction of flow of said fluid through said channel,
whereby a tactile arrow-like portion of one of said at least two connectors indicates a direction of flow towards said medical instrument, and
a tactile arrow-like portion of another one of said at least two connectors indicates a direction of flow away from said medical instrument.

2. The medical instrument of claim 1, whereby said tactile arrow-like portion is arranged on said body.

3. The medical instrument of claim 1, whereby said tactile arrow-like portion is formed by said body.

4. The medical instrument of claim 1, whereby at least one of said at least two connectors comprises a valve.

5. The medical instrument of claim 4, whereby said tactile arrow-like portion is arranged on said valve.

6. The medical instrument of claim 4, whereby said tactile arrow-like portion is formed by said valve.

7. The medical instrument of claim 1, whereby said tactile arrow-like portion comprises at least a first region and a second region,
said second region being arranged downstream of said first region in said direction of flow and
having a smaller width than said first region in at least one plane along said direction of flow.

8. The medical instrument of claim 7, whereby said tactile arrow-like portion tapers continuously from said first region to said second region.

9. The medical instrument of claim 8, whereby said tactile arrow-like portion forms an arrow.

10. The medical instrument of claim 1, whereby said tactile arrow-like portion is formed by an engraving.

11. The medical instrument of claim 1, whereby said tactile arrow-like portion is formed by an elevation.
